(19) United States
(12) Patent Application Publication
(10) Pub. No.: US 2006/0287554 A1
(43) Pub. Date: Dec. 21, 2006

(54) Process for producing inorganic salts of hop acids

(76) Inventors: Kevin K. Madsen, Yakima, WA (US); Mark M. Bossert, Yakima, WA (US); Mitsunori Ono, Lexington, MA (US)

Correspondence Address: EDWARDS & ANGELL, LLP, P.O. BOX 55874, BOSTON, MA, 02205, US

(21) Appl. No.: 11/302,308

(22) Filed: Apr. 24, 2006

Related U.S. Application Data
(60) Provisional application No. 60/692,746, filed on Jun. 21, 2005.
(60) Provisional application No. 60/692,910, filed on Jun. 21, 2005.
(60) Provisional application No. 60/749,966, filed on Dec. 12, 2005.

(51) Int. Cl. C07C 61/06; C07C 45/61

(52) U.S. Cl. 562/503; 568/379

(57) ABSTRACT

The invention relates to a novel process for preparing hops compounds and compositions produced by this process.

### RELATED APPLICATIONS

This application claims the benefit of the following U.S. Provisional Applications: Ser. No. 60/692,746, filed Jun. 21, 2005; Ser. No. 60/692,910 filed Jun. 21, 2005 and Serial No. (Not Yet Assigned), which is entitled Aqueous Compositions of Hop Acids and Uses Thereof, filed Dec. 12, 2005, the entire contents of each of which is incorporated herein by reference.

### BACKGROUND

Hops have been used for centuries to flavor beer and are considered, along with water, yeast and malt, to be an essential ingredient of beer. A goal of present brewing technology is to make reproducible brews. Compositions and methods that improve the reproducibility of hop flavors are useful for controlling and standardizing the flavoring of beer and ale.

Previous methods for producing solid salts of hop acids, such as those described in U.S. Pat. No. 5,624,701, require a four step process that includes heating an aqueous alkaline solution of a hop acid with an aqueous salt solution to produce a solid salt of a hop acid. This heating step accelerates the degradation of alpha acids during salt formation and is undesirable. In addition, this process uses hop acids at concentrations between 4% and 7% during the magnesium salt formation reaction. These low concentrations of hop acids increase the time required for the reaction, which increases costs. Improved methods of converting hop acids to solid salts of hop acids are required.

### SUMMARY

The invention relates to a novel process for preparing hops compounds and compositions produced by this process.

In particular, the present invention provides novel methods having greatly improved efficiency for making isoalpha acids, reduced isoalpha acids, tetrahydroisoalpha acids, and hexahydro-isoalpha acids; as well as for the production of beta acids. In particular, these beta acids include hexahydro beta acids and tetrahydro beta acids. In one embodiment, the hop acid is a beta acid selected from the group consisting of lupulone, colupulone, adlupulone and derivatives thereof. In another embodiment, the beta acid is a hexahydro beta acids or tetrahydro beta acids.

These improved methods are 5-10 times faster than previously described methods, which advantageously reduces labor, energy, and other production costs. Another advantage of the present methods is that they do not require a heating step, but are carried out at room temperature. This reduces the degradation of hop acids during salt formation.

In one aspect, the invention features a process for the production of an inorganic salt (e.g., magnesium or calcium) of an alpha acid, reduced isoalpha acid (e.g., isoalpha acid, reduced isoalpha acid, tetrahydroisoalpha acid, or hexahydro-isoalphaacid) or a beta acid (e.g., hexahydro beta acid, tetrahydro beta acid, lupulone, colupulone, adlupulone or derivatives thereof). The method involves (a) providing an aqueous solution containing 10-50% of an isoalpha acid or reduced isoalpha acid, wherein the solution is at room temperature; (b) adding an inorganic salt to the aqueous solution with agitation to form a slurry, where the slurry is at room temperature (e.g., between 15 and 25° C.); (c) mixing until the slurry is homogeneous; and (d) drying (e.g., spray drying, vacuum drying, drum drying, pan drying, window drying, and/or freeze drying) the slurry to obtain an inorganic salt a hop acid. In one embodiment, the method further comprises the step of filtering the slurry of step (c) prior to step (d). In another embodiment, the aqueous solution is an aqueous alkaline solution.

In another aspect, the invention features a process for the production of a magnesium salt (e.g., magnesium sulfate) of a reduced isoalpha acid. The method involves (a) providing an aqueous solution containing 10-50% of a reduced isoalpha acid, where the solution is at room temperature; (b) adding an inorganic magnesium salt to the aqueous solution with agitation to form a slurry, wherein the slurry is at room temperature; (c) mixing until the slurry is homogeneous; and (d) drying the slurry to obtain a magnesium salt of a reduced isoalpha acid. In one embodiment, the method further comprises the step of filtering the slurry of step (c) prior to step (d). In another embodiment, the aqueous solution is an aqueous alkaline solution.

In yet another aspect, the invention features a process for the production of a calcium salt of a reduced isoalpha acid. The method involves (a) providing an aqueous solution containing 10-50% of a reduced isoalpha acid, where the solution is at room temperature; (b) adding an inorganic calcium salt (e.g., calcium carbonate, calcium chloride, or calcium hydroxide) to the aqueous solution with agitation to form a slurry, where the slurry is at room temperature; (c) mixing until the slurry is homogeneous; and (d) drying the slurry to obtain a calcium salt of a reduced isoalpha acid. In one embodiment, the method further comprises the step of filtering the slurry of step (c) prior to step (d). In another embodiment, the aqueous solution is an aqueous alkaline solution.

In various embodiments of the above aspects, the concentration of alpha acid or reduced isoalpha acid present in the aqueous solution is between 10% and 50% (e.g., 10%, 15%, 20%, 25%, 30%, 35%, 40%, 45%, or 50%). In other embodiments of the above aspects, the magnesium/alpha acid or reduced isoalpha acid or calcium/alpha acid or reduced isoalpha acid molar ratio is in a range between 0.3 and 0.8 (e.g., 0.3, 0.4, 0.5, 0.6, 0.7, and 0.8). In other embodiments of any of the above aspects, room temperature is between 15 and 25° C. (e.g., 15, 16, 17, 18, 19, 20, 21, 22, 23, 24, or 25° C.).

In another aspect, the invention features a reduced isoalpha acid made by the process of any of the above aspects.

In another aspect, the invention provides a process for the production of a magnesium salt of a beta acid. The method involves providing an aqueous alkaline solution containing 10-50% of a beta acid, where the solution is at room temperature; adding an inorganic magnesium salt to the aqueous alkaline solution with agitation to form a slurry, where the slurry is at room temperature; mixing until the slurry is homogeneous; and drying the slurry to obtain a magnesium salt of a beta acid. In one embodiment, the magnesium salt is magnesium sulfate. In another embodiment, the method further comprises the step of filtering the slurry of step (c) prior to step (d).

In another aspect, the invention provides a process for the production of a calcium salt of beta acid. The method involves providing an aqueous alkaline solution containing 10-50% of a beta acid, where the solution is at room temperature (e.g., between 15° C. and 25° C., including 17, 18, 19, 20, 21, 22, 23, 24, or 25° C.) adding an inorganic calcium salt to the aqueous alkaline solution with agitation to form a slurry, where the slurry is at room temperature; mixing until the slurry is homogeneous; and drying the slurry to obtain a calcium salt of a beta acid. In one embodiment, the method further comprises the step of filtering the slurry of step (c) prior to step (d). In another embodiment, the calcium salt is at least one of calcium carbonate, calcium chloride, or calcium hydroxide. In another embodiment, the concentration of a beta acid present in the aqueous alkaline solution is between 10% and 45% (e.g., any integer between 10 and 50, wherein the bottom of the range is between 10 and 49, and the top of the range is an integer between 11 and 50; exemplary integers include 10, 15, 20, 25, 30, 35, 40, or 45%); is between 15% and 45%; or is 20%. In yet another embodiment, the magnesium/beta acid or calcium/beta acid molar ratio is in a range between 0.3 and 0.8 (e.g., 0.3, 0.4, 0.5, 0.6, 0.7, or 0.8). In yet another embodiment, the drying is accomplished by a method selected from the group consisting of spray drying, vacuum drying, drum drying, pan drying, window drying and freeze drying, or any combination thereof. In still other embodiments, the beta acid is selected from the group consisting of tetrahydro beta acids, and hexahydro beta acids.

In yet another aspect, the invention provides a beta acid made by the process of any previous aspect.

In various embodiments of any of the above aspects, the aqueous solution is an aqueous alkaline solution. By “aqueous alkaline solution” is meant any solution having a basic pH, i.e., a pH greater than neutral. In general, a neutral pH is about 7. Accordingly, an aqueous alkaline solution has a pH greater than 7, for example, a pH between 7.4 and 12 (e.g., 7.4, 7.6, 7.8, 8, 9, 10, 11, or 12), inclusive.

Hop acid derivatives are compounds that are chemically derived (either through natural biosynthetic procesess (e.g., living organism metabolism (e.g., mammal, plant, bacetria)) or synthetic processes using human intervention (e.g., chemical synthesis)) from hop acids. Alpha acid derivatives (e.g., isoalpha acids, reduced isoalpha acids, tetrahydroisoalpha acids, and hexahydro-isoalphaacids) are compounds derived from alpha hop acids.

The invention also relates to a method of making a compound described herein. The method involves any reactions or reagents or processes (including extraction, isolation, purification) as delineated in the schemes or examples herein. Alternatively, the method includes taking any one of the intermediate compounds described herein and reacting it with one or more chemical reagents in one or more steps to produce a compound described herein.

In other embodiments, the compounds, compositions, and methods delineated herein are any of the compounds delineated herein or methods including them.

The details of one or more embodiments of the invention are set forth in the description below. Other features, objects, and advantages of the invention will be apparent from the description and from the claims.

### DETAILED DESCRIPTION

The present invention provides a novel process for producing one or more hop acids or hop acid derivatives.

The present invention provides novel methods having improved efficiency for making isoalpha acids, reduced isoalpha acids, tetrahydroisoalpha acids, acids and hexahydro-isoalpha acids, as well as for making beta acids, such as lupulone, colupulone, adlupulone and their derivatives. In particular embodiments, these beta acids include hexahydro beta acids and tetrahydro beta acids.

Exemplary reduced isoalpha acids that may be made by the process of the invention include, but are not limited to, any one or more of the following formulas:

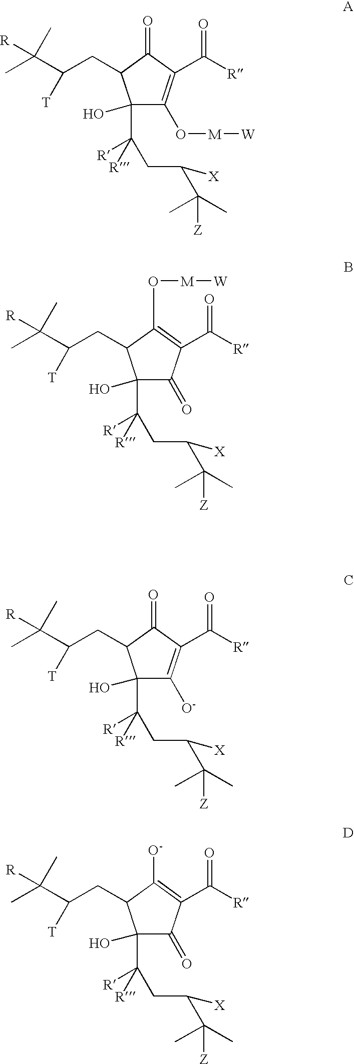

where R′ is selected from the group consisting of hydroxyl, OR and OCOR, R is independently alkyl, and R′″ is H; or R′ and R′″ taken together are ═O; and

where R″ is alkyl;

where R, T, X and Z are independently selected from the group consisting of H, F, Cl, Br, I and Pi-orbital, with the proviso that if one of R, T, X, or Z is a Pi orbital, then the adjacent R, T, X, or Z is also a Pi orbital, thereby forming a double bond;

where M is magnesium or calcium;

where W is Cl, OH, SO4-, Br, I, Formula C or Formula D.

Exemplary beta acids that may be made by the process of the invention include, but are not limited to, any one or more of the following formulas:

any one or more of Formulas A, B, C and D:

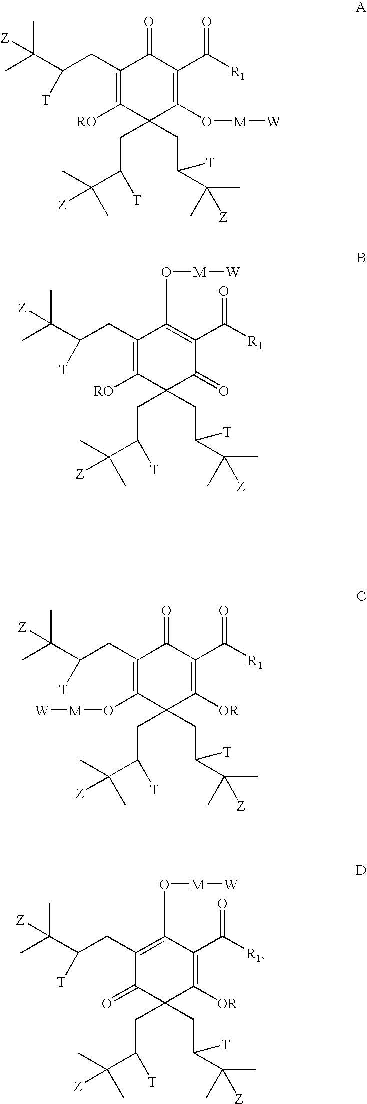

where R1 is alkyl;

where Z and T are independently selected from H and Pi-orbital with the proviso that if one of T or Z is a Pi orbital, then the adjacent T or Z is also a Pi orbital, thereby forming a double bond;

where M is magnesium or calcium;

where W is Cl, OH, SO4⁻, Br, I, or a compound of Formula E, F, G or H: 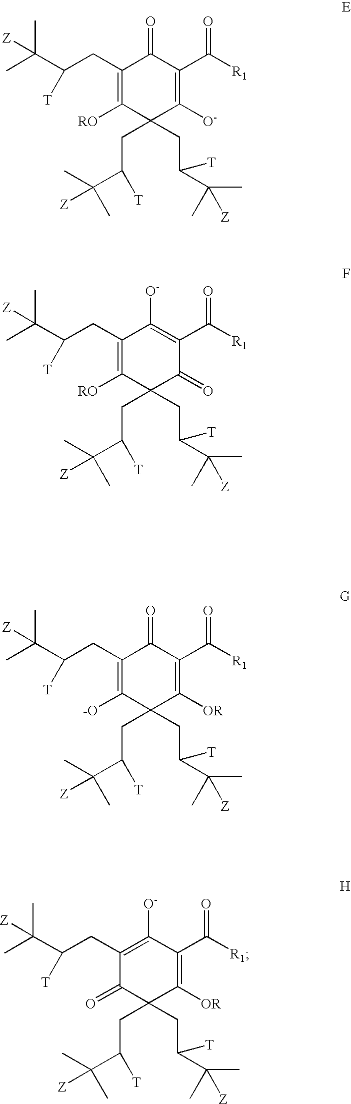
and where R is H, Na, K, Li or M-W. Advantageously, the magnesium salts of a beta acid are much less hygroscopic than the beta acids themselves.

In one aspect, the present invention provides methods for producing aqueous compositions containing between about 1% and 95%, inclusive, inorganic salts of beta acids, hexahydro beta acids and tetrahydro beta acids. Such aqueous formulations have improved bioavailability and are suitable for oral or topical administration to a subject. In addition, the production of such formulations is more efficient than prior art methods, and the aqueous formulations are more convenient to handle than prior art formulations.

As used herein, the term “isoalpha acid” refers to compounds isolated from hops plant products and which subsequently have been isomerized. The isomerization of alpha acids can occur thermally, for example, by boiling. Examples of isoalpha acids include, but are not limited to, isocohumulone, and isoadhumulone.

As used herein, the term “reduced isoalpha acid” refers to alpha acids isolated from hops plant product and which subsequently have been isomerized and reduced, including cis and trans forms. Examples of reduced isoalpha acids include, but are not limited to, dihydro-isohumulone, dihydro-isocohumulone, and dihydro-adhumulone.

As used herein, the term “tetra-hydroisoalpha acid” refers to a certain class of reduced isoalpha acid. Examples of tetra-hydroisoalpha acid include, but are not limited to, tetra-hydro-isohumulone, tetra-hydro-isocohumulone and tetra-hydro-adhumulone.

As used herein, the term “hexa-hydroisoalpha acid” refers to a certain class of reduced isoalpha acid. Examples of hexa-hydroisoalpha acids include, but are not limited to, hexa-hydro-isohumulone, hexa-hydro-isocohumulone and hexa-hydro-adhumulone.

As used herein, the term “beta acid” refers to compounds collectively known as lupulones that can be isolated from hops plant products, including but not limited to, lupulone, adlupulone, colupulone, tetrahydro lupulone, tetrahydroadlupulone, tetrahydrocolupulone and their derivatives. Exemplary beta acids include, but are not limited to, hexahydro beta acids and tetrahydro beta acids.

As used herein, the term “halo” refers to any radical of fluorine, chlorine, bromine or iodine.

The term “alkyl” refers to a hydrocarbon chain that may be a straight chain or branched chain, containing 1-20 or the indicated number of carbon atoms. For example, C₁-C₅ indicates that the group may have from 1 to 5 (inclusive) carbon atoms in it. The term “lower alkyl” refers to a C₁-C₆ alkyl chain. The term “alkenyl” refers to a hydrocarbon chain that may be a straight chain or branched chain, containing 1-20 or the indicated number of carbon atoms and one or more double bonds in the chain (e.g., propylenyl, isopentylenyl). For example, C₁-C₁₀ indicates that the group may have from 1 to 10 (inclusive) carbon atoms in it. The term “arylalkyl” refers to a moiety in which an alkyl hydrogen atom is replaced by an aryl group. The term “cycloalkylalkyl” refers to a moiety in which an alkyl hydrogen atom is replaced by a cycloalkyl group.

The term “aryl” refers to an aromatic monocyclic, bicyclic, or tricyclic ring system having carbon ring atoms, wherein 0, 1, 2, 3, or 4 atoms of each ring may be substituted by a substituent.

The term “cycloalkyl” as employed herein includes saturated and partially unsaturated cyclic hydrocarbon groups having 3 to 12 carbons, preferably 3 to 8 carbons, and more preferably 3 to 6 carbons.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

As used herein the term “substituent” or “substituted” means that a hydrogen radical on a compound or group (such as, for example, alkyl, alkenyl, alkynyl, alkylene, aryl, aralkyl, heteroaryl, heteroaralkyl, cycloalkyl, cyclyl, heterocycloalkyl, or heterocyclyl group) is replaced with any desired group that do not substantially adversely affect the stability of the compound. In one embodiment, desired substituents are those which do not adversely affect the activity of a compound. The term “substituted” refers to one or more substituents (which may be the same or different), each replacing a hydrogen atom.

Examples of substituents include, but are not limited to, halogen (F, Cl, Br, or I), hydroxyl, amino, alkylamino, arylamino, dialkylamino, diarylamino, cyano, nitro, mercapto, oxo (i.e., carbonyl), thio, imino, formyl, carbamido, carbamyl, carboxyl, thioureido, thiocyanato, sulfoamido, sulfonylalkyl, sulfonylaryl; alkyl, alkenyl, alkoxy, mercaptoalkoxy, aryl, heteroaryl, cyclyl, heterocyclyl, wherein alkyl, alkenyl, alkyloxy, aryl, heteroaryl, cyclyl, and heterocyclyl are optionally substituted with alkyl, aryl, heteroaryl, halogen, hydroxyl, amino, mercapto, cyano, nitro, oxo (═O), thioxo (═S), or imino (═NR), where R is as defined herein.

Reduced isoalpha acids can be prepared by purification from natural hops and also chemical synthesis according to traditional methods.

The term “extract” refers to a concentrated preparation of the essential constituents of a plant (e.g., medicinal plant, hops). Typically, an extract is prepared by drying and powderizing the plant. Optionally, the plant, the dried plant or the powderized plant may be boiled in solution. The extract may be used in liquid form, or it may be mixed with other liquid or solid herbal extracts. Alternatively, the herbal extract may be obtained by further precipitating solid extracts from the liquid form. Edible plant extracts include those from any plant that is edible to a human (e.g., fruit extract, vegetable extract, root extract, leaf extract, tree or bark extract, bean extract, and the like) and includes, for example, green tea extract, red onion extract, grape seed extract, cocoa extract, red clover extracts, and soy extracts.

An extract can be prepared by drying and subsequently cutting or grinding the dried material. The extraction process may then be performed with the help of an appropriate choice of solvent, typically ethanol/water mixture, methanol, butanol, iso-butanol, acetone, hexane, petroleum ether or other organic solvents by means of maceration, percolation, repercolation, counter-current extraction, turbo-extraction, or by carbon-dioxide hypercritical (temperature/pressure) extraction. The extract may then be further evaporated and thus concentrated to yield by means of air drying, spray drying, vacuum oven drying, fluid-bed drying or freeze-drying, the extract product.

The synthesized compounds can be separated from a reaction mixture and further purified by a method such as column chromatography, high pressure liquid chromatography, or recrystallization. As can be appreciated by the skilled artisan, further methods of synthesizing the compounds herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 2nd. Ed., Wiley-VCH Publishers (1999); T. W. Greene and P. G. M. Wuts, Protective Groups in Organic Synthesis, 3rd. Ed., John Wiley and Sons (1999); L. Fieser and M. Fieser, Fieser and Fieser's Reagents for Organic Synthesis, John Wiley and Sons (1999); and L. Paquette, ed., Encyclopedia of Reagents for Organic Synthesis, John Wiley and Sons (1995), and subsequent editions thereof.

The compounds of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also be represented in multiple tautomeric forms, in such instances; the invention expressly includes all tautomeric forms of the compounds described herein. All such isomeric forms of such compounds are expressly included in the present invention. All crystal forms of the compounds described herein are expressly included in the present invention.

The invention will be further described in the following examples. It should be understood that these examples are for illustrative purposes only and are not to be construed as limiting this invention in any manner.

### EXAMPLES

1. Preparation of a Reduced Isoalpha Acid Salts

An inorganic salt of a reduced isoalpha acid is produced using any standard methods known in the art. In one embodiment, a reduced isoalpha acid is produced according to the following method.

An empty drum was placed on a scale and tared. To the drum was added 80 kg of a mixture of reduced isohops (30%) in deionized water (75 L) at room temperature. The mixture was subjected to gentle agitation to form an aqueous slurry. MgSO4 (45 kg) was added to the slurry at one time and the agitation was continued for 5-10 minutes until the MgSO4 was homogeneously distributed. After 10 minutes, a small sample was removed to determine whether the reaction had reached completion. This was determined using an HPLC to assay the presence of reduced isoalpha magnesium salt. When the reaction was complete, the mixture was removed and deionized water was added to adjust the concentration of reduced isoalpha magnesium salt to 15-17% having 83-85% water content. The mixture was then dried using standard methods. When the drying was completed, the flaky products were packed in aluminum coated polyethelene bags, heat sealed and stored at room temperature prior to analysis.

2. Preparation of Beta Acid Magnesium Salts

An inorganic salt of a beta acid is produced using any standard methods known in the art. In one embodiment, a beta acid is produced according to the following method.

Beta acid magnesium salts were prepared as follows. 5000 ml of hop beta acid solution containing approximately 500 g of beta acid potassium salt was stirred at room temperature. The pH of the solution was adjusted to pH 11.50 by the drop wise addition of 100 ml of 20% KOH solution. The solution was then diluted with deionized water (1100 ml) while the pH was maintained at 11.50.

840 ml of 10% MgSO4 solution was added to the solution under vigorous stirring at room temperature. After the addition, the mixture was stirred for 30 minutes then the white precipitate was filtered through a Buchner funnel using Whatman #45 filter paper. The precipitate was washed with deionized water and dried to get 490 g of beta acid magnesium salt (purity: >95% as the magnesium content).

5000 ml of hop beta acid solution that contained approximately 500 g of beta acid potassium salt was stirred at room temperature. The solution's pH was adjusted to pH 11.50 by the drop wise addition of 100 ml of 20% KOH solution. The solution was then diluted with deionized water (1100 ml) while the pH was maintained at 11.50.

840 ml of 10% of MgSO4 solution was added to the solution under vigorous stirring at room temperature. After the addition, the mixture was stirred for 30 minutes then the mixture was evaporated to obtain 560 g of a pale yellow solid. (purity: >70%).

3. Preparation of Tetrahydrobeta Acid Magnesium Salts

Tetrahydrobeta acid magnesium salts were obtained as follows. 1.25 liters of an aqueous alkaline solution containing 20% tetrahydrobeta acids was blended with 1.25 liter of an aqueous 6M potassium carbonate solution at room temperature. After stirring for 30 minutes, the solution's pH was adjusted to pH 11.50 by the drop wise addition of 50 ml of 20% KOH solution. The solution was then diluted with deionized water (550 ml) while maintaining the solution pH at 11.50. 420 ml of 10% of MgSO4 solution was added to the solution under vigorous stirring at room temperature. After the addition, the mixture was stirred for 30 minutes then the white precipitate was filtered through a Buchner funnel using Whatman #45 filter paper and washed with deionized water and dried to get 220 g of beta acid magnesium salt (purity: >95% as the magnesium content).

Virtually any magnesium salt may be used in the process set forth above. In one embodiment, the magnesium salt is magnesium sulfate. In yet other embodiments of any of the above aspects, the magnesium/reduced isoalpha acid or calcium/reduced isoalpha acid molar ratio is in a range between 0.3 and 0.8 (e.g., 0.3, 0.4, 0.5, 0.6, 0.7, 0.8), inclusive. In one embodiment of any of the above aspects, the magnesium/reduced isoalpha acid or calcium/reduced isoalpha acid molar ratio is in a range between 0.4 and 0.6. In related embodiments, the lower limit of the range is any number between 0.3 and 0.79 and the upper limit of the range is any number between 0.35 and 0.8.

As set forth in the above examples, the invention provides processes for producing a solid salt of a reduced isoalpha acid. Virtually any isoalpha acid, reduced isoalpha acid, tetrahydroisoalpha acid, and hexahydro-isoalpha acid may be used in the processes of the invention. In one embodiment, the concentration of a reduced isoalpha acid present in the aqueous solution ranges between 10% and 50%, inclusive. In other embodiments, the concentration ranges between 15-45% (e.g., 15%, 20%, 25%, 30%, 40%, and 45%), inclusive. In yet other embodiments, the lower end of the range is any number between 10 and 49%; and the upper end of the range is any number between 11 and 50%. The slurry may be dried to obtain an inorganic salt (e.g., magnesium or calcium) of a reduced isoalpha acid using any standard method or combination of methods, including but not limited to, spray drying, vaccum drying, drum drying, pan drying, window drying and freeze drying.

This process provides advantages over previous methods for producing solid salts of hop acids, such as those described in U.S. Pat. No. 5,624,701, which require a four step process that includes heating an aqueous alkaline solution of a hop acid with an aqueous salt solution to produce a solid salt of a hop acid. This heating step accelerates the degradation of alpha acids during salt formation and is undesirable. Advantageously, the present method does not require a heating step, but is carried out at room temperature. The term “room temperature” means between 15° C. and 25° C. (e.g., 15, 16, 17, 18, 19, 20, 21, 22, 23, 24, or 25° C.), where the lower end of the range is any number between 15 and 24; and the upper end of the range is any number between 16 and 25.

In addition, prior methods required using hop acids at concentrations between 4% and 7% inclusive during the magnesium salt formation reaction. These low concentrations of hop acids increase the time required for the reaction, and increase costs. The present invention uses reduced isoalpha acid at concentrations between 10% and 50% (e.g., 10, 15, 20, 25, 30, 35, 45, and 50%), inclusive. These higher concentrations allow the reaction to proceed 5-10 times more quickly than previously described methods, which advantageously reduces labor, energy, and other production costs.

Compounds are prepared in a manner essentially as described above and in the general schemes. The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. Another embodiment is a compound of any of the formulae herein made by a process delineated herein, including the processes exemplified in the schemes and examples herein. Another aspect of the invention is a compound of any of the formulae herein for use in the treatment or prevention in a subject of a disease, disorder or symptom thereof delineated herein. Another aspect of the invention is use of a compound of any of the formulae herein in the manufacture of a medicament for treatment or prevention in a subject of a disease, disorder or symptom thereof delineated herein.

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A process for the production of an inorganic salt of a hop acid, the method comprising:
(a) providing an aqueous solution containing 10-50% of a hop acid, wherein the solution is at room temperature;
(b) adding an inorganic salt to the aqueous solution with agitation to form a slurry, wherein the slurry is at room temperature;
(c) mixing until the slurry is homogeneous; and
(d) drying the slurry to obtain an inorganic salt of a hop acid.

2. (canceled)

3. The process of claim 1, wherein the inorganic salt is magnesium or calcium.

4. The process of claim 1, wherein the hop acid is an isoalpha acid is selected from the group consisting of isoalpha acid, reduced isoalpha acid, tetrahydroisoalpha acid, and hexahydro-isoalpha acid.

5. The process of claim 1, wherein the hop acid is a beta acid selected from the group consisting of lupulone, colupulone, adlupulone and derivatives thereof.

6. The process of claim 5, wherein the beta acid is a hexahydro beta acids or tetrahydro beta acids.

7. The process of claim 1, wherein the method further comprises the step of filtering the homogenous slurry of step (c) prior to step (d).

8. A process for the production of a magnesium or calcium salt of an isoalpha acid or a reduced isoalpha acid, the method comprising:
(a) providing an aqueous solution containing 10-50% of an isoalpha acid or a reduced isoalpha acid, wherein the solution is at room temperature;
(b) adding an inorganic magnesium or calcium salt to the aqueous alkaline solution with agitation to form a slurry, wherein the slurry is at room temperature;
(c) mixing until the slurry is homogeneous; and
(d) drying the slurry to obtain a magnesium or calcium salt of an isoalpha acid or a reduced isoalpha acid.

9. The process of claim 8, wherein the aqueous solution is an aqueous alkaline solution.

10. The process of claim 8, wherein the magnesium salt is magnesium sulfate.

11-12. (canceled)

13. The process of claim 8, wherein the calcium salt is at least one of calcium carbonate, calcium chloride, or calcium hydroxide.

14. The process of claim 8, wherein the concentration of an isoalpha acid or of a reduced isoalpha acid present in the aqueous solution is between 10% and 45%.

15. (canceled)

16. The process of claim 8, wherein the concentration of an isoalpha acid or of a reduced isoalpha acid present in the aqueous solution is 15%.

17. The process of claim 8, wherein the magnesium/isoalpha acid or reduced isoalpha acid or calcium/isoalpha acid or reduced isoalpha acid molar ratio is in a range between 0.3 and 0.8.

18. The process of claim 8, wherein the drying is accomplished by a method selected from the group consisting of spray drying, vacuum drying, drum drying, pan drying, window drying and freeze drying, or any combination thereof.

19. The process of claim 8, wherein the reduced isoalpha acid is selected from the group consisting of tetrahydroisoalpha acids and hexahydro-isoalpha acids.

20. (canceled)

21. The process of claim 8, wherein the method further comprises the step of filtering the homogenous slurry of step (c) prior to step (d).

22. A reduced isoalpha acid made by the process of claim 8.

23. A process for the production of a magnesium or calcium salt of an beta acid, the method comprising:
(a) providing an aqueous alkaline solution containing 10-50% of a beta acid, wherein the solution is at room temperature;
(b) adding an inorganic magnesium or calcium salt to the aqueous alkaline solution with agitation to form a slurry, wherein the slurry is at room temperature;
(c) mixing until the slurry is homogeneous; and
(d) drying the slurry to obtain a magnesium or calcium salt of a beta acid.

24. The process of claim 23, wherein the magnesium salt is magnesium sulfate.

25. (canceled)

26. The process of claim 23, wherein the calcium salt is at least one of calcium carbonate, calcium chloride, or calcium hydroxide.

27. The process of claim 23, wherein the concentration of a beta acid present in the aqueous alkaline solution is between 10% and 45%.

28. (canceled)

29. (canceled)

30. The process of claim 23, wherein the magnesium/beta acid or calcium/beta acid molar ratio is in a range between 0.3 and 0.8.

31. The process of claim 23, wherein the method further comprises the step of filtering the homogenous slurry of step (c) prior to step (d).

32. The process of claim 23, wherein the drying is accomplished by a method selected from the group consisting of spray drying, vacuum drying, drum drying, pan drying, window drying and freeze drying, or any combination thereof.

33. The process of claim 23, wherein the beta acid is selected from the group consisting of tetrahydro beta acids, and hexahydro beta acids.

34. (canceled)

35. A beta acid made by the process of claim 23.
